(19) United States
(12) Patent Application Publication
(10) Pub. No.: US 2021/0244799 A1
(43) Pub. Date: Aug. 12, 2021

(54) USES OF GLUCAGON-LIKE PEPTIDE-1 RECEPTOR AGONISTS FOR TREATING TRAUMA-INDUCED HEARING LOSS

(71) Applicant: The Board of Regents of the University of Oklahoma, Norman, OK (US)

(72) Inventors: Rong Z. Gan, Oklahoma City, OK (US); Tao Chen, Chongqing (CN)

(21) Appl. No.: 17/231,381

(22) Filed: Apr. 15, 2021

Related U.S. Application Data
(63) Continuation-in-part of application No. PCT/US2019/054983, filed on Oct. 7, 2019.
(60) Provisional application No. 62/746,055, filed on Oct. 16, 2018.

(51) Int. Cl. A61K 38/26; A61P 27/16

(52) U.S. Cl. CPC A61K 38/26; A61P 27/16

(57) ABSTRACT

Methods of treating a subject for hearing loss or auditory impairment or damage, such as blast-induced hearing damage, comprising administering to the subject in need of such treatment an effective quantity of a glucagon-like peptide-1 receptor (GLP-1R) agonist. The treatment may be given after exposure to a blast, loud noise, or other hearing loss-inducing traumatic event, or may be prophylactic, i.e., given prior to exposure to a blast or loud noise.

### CROSS-REFERENCE TO RELATED APPLICATIONS

The present application continuation-in-part national stage filing of PCT Application No. PCT/US2019/054983, filed Oct. 7, 2019, which claims benefit under 35 USC § 119(e) of U.S. Provisional Application Ser. No. 62/746,055, filed Oct. 16, 2018, both of which are hereby expressly incorporated herein by reference in their entireties.

### GOVERNMENT SUPPORT

This invention was made with government support under U.S. Department of Defense grant W81XWH-14-1-0228. The government has certain rights in the invention.

### BACKGROUND

Blast overpressure (BOP) is a high intensity disturbance in the ambient air pressure. When exposed to blast, the human auditory system is vulnerable to both peripheral (middle ear and cochlea) and central damage (brainstem and brain) from the BOP. Repetitive blast exposures, for example, 3 consecutive exposures on Day 1 (3-Blast), even at a low overpressure level (below mild traumatic brain injury (mTBI)) frequently result in permanent hearing damage in Service members. Recent studies have assessed the Incretin glucagon-like peptide-1 receptor (GLP-1R) agonist Liraglutide as a potential treatment strategy for memory impairment and cognitive deficits due to mild to moderate TBI (Li et al., Liraglutide is neurotrophic and neuroprotective in neuronal cultures and mitigates mild traumatic brain injury in mice. J. Neurochemistry, Vol. 135(6): 1203-1217, 2015; and Tweedie et al., Blast traumatic brain injury-induced cognitive deficits are attenuated by pre-injury or post-injury treatment with the glucagon-like peptide-1 receptor agonist, exendin-4. Alzheimer's & Dementia, Vol. 12(1): 34-48, 2016).

### BRIEF DESCRIPTION OF THE DRAWINGS

FIG. 1A shows micrographs of a chinchilla cochlear structure immunohistochemically (IHC) stained to show expression of GLP-1R.

FIG. 1B shows micrographs of chinchilla cochlea immunohistochemically (IHC) stained to show expression of GLP-1R in spiral ganglion neurons of the cochlear base turn.

FIG. 2 shows micrographs of portions of the chinchilla central auditory system immunohistochemically (IHC) stained to show expression of GLP-1R. GLP-1R expression in the auditory cortex is shown at various magnifications in the top three images. GLP-1R expression in the inferior colliculus is shown at various magnifications in the bottom three images. Arrows mark the GLP-1R expression in auditory cortex and inferior colliculus.

FIG. 3A shows the mean and SD of the ABR threshold across the frequency range of 0.5-8 kHz obtained from the control animals (n=10 ears, upper panel) and pre-treatment animals (n=8 ears, lower panel) after exposure to three blasts at BOP of 20 psi on Day 1.

FIG. 3B shows the mean and SD of the ABR threshold shift across the frequency range of 0.5-8 kHz obtained from the control animals (n=10 ears, upper panel) and pre-treatment animals (n=8 ears, lower panel) after exposure to three blasts at BOP of 20 psi on Day 1.

FIG. 4A shows a comparison of the ABR threshold shifts between the pre-treatment and control animals measured at 4 hours on Day 1 (upper panel), and 4 days (lower panel) after exposure to three blasts at BOP level of 20 psi. The data are plotted as group means±SD. n represents the number of ears per group.

FIG. 4B shows a comparison of the ABR threshold shifts between the pre-treatment and control animals measured at 7 days (upper panel) and 14 days (lower panel) after exposure to three blasts at BOP level of 20 psi. The data are plotted as group means±SD. n represents the number of ears per group.

FIG. 5 shows a comparison of the ABR threshold (upper panel) and threshold shift (lower panel) averaged over tested frequencies (0.5-8 kHz) between the pre-treatment and control animals during 14 days after exposure to three times of BOP at 20 psi on Day 1. The data are plotted as group means±SD. n represents the number of ears per group.

### DETAILED DESCRIPTION

The present disclosure is directed to, in at least certain embodiments, uses of glucagon-like peptide-1 receptor (GLP-1R) agonists for restoration of hearing loss or auditory impairment or damage. In certain embodiments, the hearing loss or auditory impairment or damage is blast-induced or noise-induced, such as caused by bomb explosions, or other loud noises. As noted above, GLP-1R agonists (also referred to herein as GLP-1 agonists and GLP-1 analogs) have been suggested as a potential treatment strategy for memory impairment and cognitive deficits due to mild to moderate traumatic brain injury (TBI). However, heretofore neither GLP-1 or GLP-1R has been reported as having therapeutic involvement benefit in treating auditory impairment such as blast-induced progressive hearing damage. Work shown herein demonstrates that GLP-1R agonists can be used therapeutically to at least partially restore blast-induced acute and progressive hearing damage. Examples of such GLP-1R agonists include, but are not limited to, liraglutide and derivatives, exenatide and derivatives, exendin-4 and derivatives, dulaglutide and derivatives, semaglutide and derivatives, albiglutide and derivatives, lixisenatide and derivatives, taspoglutide and derivatives, and GLP-1 agonists and analogs thereof as disclosed in U.S. Published Patent Applications 2015/0038417, 2016/0347813, 2018/0085435, 2018/0280481, and 2018/0000903.

Before further detailed description of various embodiments of the compositions and methods of use thereof of the present disclosure, it is to be understood that the present disclosure is not limited in application to the details of methods and compositions as set forth in the following description. The description provided herein is intended for purposes of illustration only and is not intended to be construed in a limiting sense. The present disclosure is capable of other embodiments or of being practiced or carried out in various ways. As such, the language used herein is intended to be given the broadest possible scope and meaning; and the embodiments are meant to be exemplary, not exhaustive. Also, it is to be understood that the phraseology and terminology employed herein is for the purpose of description and should not be regarded as limiting unless otherwise indicated as so. Moreover, in the following detailed description, numerous specific details are set forth in order to provide a more thorough understanding of the disclosure. However, it will be apparent to a person having ordinary skill in the art that various embodiments of the present disclosure may be practiced without these specific details. In other instances, features which are well known to persons of ordinary skill in the art have not been described in detail to avoid unnecessary complication of the description. It is intended that all alternatives, substitutions, modifications and equivalents apparent to those having ordinary skill in the art are included within the scope of the present disclosure as defined herein. Thus the examples described below, which include particular embodiments, will serve to illustrate the practice of the present disclosure, it being understood that the particulars shown are by way of example and for purposes of illustrative discussion of particular embodiments only and are presented in the cause of providing what is believed to be a useful and readily understood description of procedures as well as of the principles and conceptual aspects of the inventive concepts. Thus, while the compositions and methods of the present disclosure have been described in terms of particular embodiments, it will be apparent to those of skill in the art that variations may be applied to the compositions and/or methods and in the steps or in the sequence of steps of the method described herein without departing from the concept, spirit, and scope of the inventive concepts disclosed herein.

All patents, published patent applications, and non-patent publications mentioned in the specification are indicative of the level of skill of those skilled in the art to which the present disclosure pertains. Each patent, published patent application, and non-patent publication referenced in any portion of this application (e.g., U.S. Published Patent Applications 2015/0038417, 2016/0347813, 2018/0085435, 2018/0280481, and 2018/0000903) is expressly incorporated herein by reference in its entirety to the same extent as if the individual patent, or published patent application, or non-patent publication was specifically and individually indicated to be incorporated by reference.

Unless otherwise defined herein, scientific and technical terms used in connection with the present disclosure shall have the meanings that are commonly understood by those having ordinary skill in the art. Further, unless otherwise required by context, singular terms shall include pluralities and plural terms shall include the singular.

As utilized in accordance with the methods and compositions of the present disclosure, the following terms, unless otherwise indicated, shall be understood to have the following meanings:

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or when the alternatives are mutually exclusive, although the disclosure supports a definition that refers to only alternatives and “and/or.” The use of the term “at least one” will be understood to include one as well as any quantity more than one, including but not limited to, 2, 3, 4, 5, 6, 7, 8, 9, 10, 15, 20, 30, 40, 50, 100, or any integer inclusive therein. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y and Z” will be understood to include X alone, Y alone, and Z alone, as well as any combination of X, Y and Z.

As used in this specification and claims, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

Throughout this application, the terms “about” or “approximately” are used to indicate that a value includes the inherent variation of error for the composition, the method used to administer the composition, or the variation that exists among the study subjects. As used herein the qualifiers “about” or “approximately” are intended to include not only the exact value, amount, degree, orientation, or other qualified characteristic or value, but are intended to include some slight variations due to measuring error, manufacturing tolerances, observer error, and combinations thereof, for example. The terms “about” or “approximately”, where used herein when referring to a measurable value such as an amount, a temporal duration, and the like, are meant to encompass, for example, variations of ±20% or ±10%, or ±5%, or ±1%, or ±0.1% from the specified value, as such variations are appropriate to perform the disclosed methods and as understood by persons having ordinary skill in the art. As used herein, the term “substantially” means that the subsequently described event or circumstance completely occurs or that the subsequently described event or circumstance occurs to a great extent or degree. For example, the term “substantially” means that the subsequently described event or circumstance occurs at least 80% of the time, at least 90% of the time, at least 91% of the time, at least 92% of the time, at least 93% of the time, at least 94% of the time, at least 95% of the time, at least 96% of the time, at least 97% of the time, at least 98% of the time, or at least 99% of the time.

As used herein any reference to “one embodiment” or “an embodiment” means that a particular element, feature, composition, structure, or characteristic described in connection with the embodiment is included in at least one embodiment. The appearances of the phrase “in one embodiment” in various places in the specification are not necessarily all referring to the same embodiment.

The term “active agent” where used herein refers to a GLP-1R agonist, GLP-1 agonist, or GLP-1 analog that has agonistic activity toward the GLP-1 receptor.

Noise-induced hearing loss (NIHL) occurs as a result of a short exposure to an intense noise (impulse or blast). In certain embodiments, the NIHL may be an acute acoustic trauma (“sudden hearing loss”) caused by an impulse or blast having a sound pressure level (SPL) of 100 dB or greater (e.g., at least 100 dB, at least 110 dB, at least 120 dB, at least 130 dB, at least 140 dB, at least 150 dB, or at least 160 dB). Or the NIHL may be a result of continuous exposure to loud sounds at or above 85 dB SPL for a relatively long period of time (“chronic NIHL”). Blast and impulse noise both can be characterized as to source of the pressure and waveform characteristics. However, blast noise is differentiated from impulse noise based the following. First, peak overpressures of blasts generate tens of kPa, whereas impulse noise overpressures are generally less than 160 dB (−2 kPa). Second, blasts produce considerable amounts of air movement and combustion products, whereas impulse noises do not. Third, impulse noise is generally related with low frequency mechanical clatter.

Blast refers to a high intensity disturbance in ambient air pressure, called blast overpressure, which occurs when solids or liquids are rapidly converted into gas. In the military, blast overpressure typically occurs from muzzle blast when heavy weapons are fired, or from the detonation of explosives (e.g., IEDs) and munitions (e.g., incoming artillery rounds). A blast may be an explosion (for example from an improvised explosive device (IED)) having sufficient concussive force to cause a TBI in a subject exposed to the blast. Blast-induced hearing damage refers to acute acoustic trauma or progressive hearing loss or damage in a subject that is due to a blast to which a subject was exposed.

The terms “peptide” where used herein may refer to a molecule comprising only amino acids, or may refer to a molecule comprising amino acids and one or more non-amino acid structures (e.g., PEG units). The terms “peptide analog”, “peptide derivative”, or “peptide compound”, may refer to a variant (“mutant”) of a “wild-type” peptide, or to a molecule comprising amino acids and one or more non-amino acid structures (e.g., PEG units).

The term “pharmaceutically acceptable” refers to compounds and compositions which are suitable for administration to humans and/or animals without undue adverse side effects such as toxicity, irritation and/or allergic response commensurate with a reasonable benefit/risk ratio.

By “biologically active” is meant the ability to modify the physiological system of an organism without reference to how the active agent has its physiological effects.

As used herein, “pure,” or “substantially pure” means an object species is the predominant species present (i.e., on a molar basis it is more abundant than any other object species in the composition thereof), and particularly a substantially purified fraction is a composition wherein the object species comprises at least about 50 percent (on a molar basis) of all macromolecular species present. Generally, a substantially pure composition will comprise more than about 80% of all macromolecular species present in the composition, more particularly more than about 85%, more than about 90%, more than about 95%, or more than about 99%. The term “pure” or “substantially pure” also refers to preparations where the object species (e.g., the peptide compound) is at least 60% (w/w) pure, or at least 70% (w/w) pure, or at least 75% (w/w) pure, or at least 80% (w/w) pure, or at least 85% (w/w) pure, or at least 90% (w/w) pure, or at least 92% (w/w) pure, or at least 95% (w/w) pure, or at least 96% (w/w) pure, or at least 97% (w/w) pure, or at least 98% (w/w) pure, or at least 99% (w/w) pure, or 100% (w/w) pure.

The terms “subject” and “patient” are used interchangeably herein and will be understood to refer to a warm blooded animal, particularly a mammal. Non-limiting examples of animals within the scope and meaning of this term include dogs, cats, rabbits, rats, mice, guinea pigs, chinchillas, hamsters, ferrets, horses, pigs, goats, cattle, sheep, zoo animals, camels, llamas, non-human primates, including Old and New World monkeys and non-human primates (e.g., cynomolgus macaques, chimpanzees, rhesus monkeys, orangutans, and baboons), and humans.

“Treatment” refers to therapeutic treatments. “Prevention” refers to prophylactic or preventative treatment measures taken prior to a damage-inducing effect or episode, such as a blast due to an explosion. The term “treating” refers to administering the composition to a patient for therapeutic or preventative purposes.

The terms “therapeutic composition” and “pharmaceutical composition” refer to an active agent-containing composition that may be administered to a subject by any method known in the art or otherwise contemplated herein, wherein administration of the composition brings about a therapeutic effect as described elsewhere herein. In addition, the compositions of the present disclosure may be designed to provide delayed, controlled, extended, and/or sustained release using formulation techniques which are well known in the art.

The term “effective amount” refers to an amount of an active agent which is sufficient to exhibit a detectable therapeutic effect without excessive adverse side effects (such as toxicity, irritation and allergic response) commensurate with a reasonable benefit/risk ratio when used in the manner of the inventive concepts. The effective amount for a patient will depend upon the type of patient, the patient's size and health, the nature and severity of the condition to be treated, the method of administration, the duration of treatment, the nature of concurrent therapy (if any), the specific formulations employed, and the like. Thus, it is not possible to specify an exact effective amount in advance. However, the effective amount for a given situation can be determined by one of ordinary skill in the art using routine experimentation based on the information provided herein.

The term “ameliorate” means a detectable or measurable improvement in a subject's condition, disease or symptom thereof. A detectable or measurable improvement includes a subjective or objective decrease, reduction, inhibition, suppression, limit or control in the occurrence, frequency, severity, progression, or duration of the condition or disease, or an improvement in a symptom or an underlying cause or a consequence of the disease, or a reversal of the disease. A successful treatment outcome can lead to a “therapeutic effect,” or “benefit” of ameliorating, decreasing, reducing, inhibiting, suppressing, limiting, controlling or preventing the occurrence, frequency, severity, progression, or duration of a disease or condition, or consequences of the disease or condition in a subject.

A decrease or reduction in worsening, such as stabilizing the condition or disease, is also a successful treatment outcome. A therapeutic benefit therefore need not be complete ablation or reversal of the disease or condition, or any one, most or all adverse symptoms, complications, consequences or underlying causes associated with the disease or condition. Thus, a satisfactory endpoint may be achieved when there is an incremental improvement such as a partial decrease, reduction, inhibition, suppression, limit, control, or prevention in the occurrence, frequency, severity, progression, or duration, or inhibition or reversal of the condition or disease (e.g., stabilizing), over a short or long duration of time (hours, days, weeks, months, etc.). Effectiveness of a method or use, such as a treatment that provides a potential therapeutic benefit or improvement of a condition or disease, can be ascertained by various methods and testing assays.

The term “peptide” is used herein to designate a series of amino acid residues, connected one to the other typically by peptide bonds between the alpha-amino and carbonyl groups of the adjacent amino acids to form an amino acid sequence. In certain embodiments, the peptides can range in length from 5 to 15 to 25 to 40 to 60 to 75 amino acids, for example, 5, 6, 7, 8, 9, 10, 11, 12, 13, 14, 15, 16, 17, 18, 19, 20, 21, 22, 23, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 to 75 amino acids. The term “polypeptide” or “protein” is used herein to designate a series of amino acid residues, connected one to the other typically by peptide bonds between the alpha-amino and carbonyl groups of the adjacent amino acids, wherein the length is longer than a single peptide. A “fusion protein” or “fusion polypeptide” refers to proteins or polypeptides (and may be used interchangeably) which have been created by recombinant or synthetic methods to combine peptides in a serial configuration. In general, the active agents of the present disclosure are peptides.

Peptides of the present disclosure and the nucleic acids which encode them include peptide and nucleic acid variants which comprise substitutions (conservative or non-conservative) of the native amino acids or bases. For example, the peptide variants include, but are not limited to, variants that are not exactly the same as the sequences disclosed herein, but which have, in addition to the substitutions explicitly described for various sequences listed herein, additional substitutions of amino acid residues (conservative or non-conservative) which substantially do not impair the activity or properties of the variants described herein. Examples of such conservative amino acid substitutions may include, but are not limited to, ala to gly, ser, or thr; arg to gln, his, or lys; asn to asp, gln, his, lys, ser, or thr; asp to asn or glu; cys to ser; gln to arg, asn, glu, his, lys, or met; glu to asp, gln, or lys; gly to pro or ala; his to arg, asn, gln, or tyr; ile to leu, met, or val; leu to ile, met, phe, or val; lys to arg, asn, gln, or glu; met to gln, ile, leu, or val; phe to leu, met, trp, or tyr; ser to ala, asn, met, or thr; thr to ala, asn, ser, or met; trp to phe or tyr; tyr to his, phe or trp; and val to ile, leu, or met.

One of ordinary skill in the art would readily know how to make, identify, select or test such variants for binding activity against the same receptors targeted by the non-variant peptides. Particular examples of conservative amino acid substitutions include, but are not limited to, gly:ala substitutions; val:ile:leu substitutions; asn:glu:his substitutions; asp:glu substitutions; ser:thr:met substitutions; lys:arg:his substitutions; and phe:tyr:trp substitutions. Other types of substitutions, variations, additions, deletions and derivatives that result in functional variant peptides are also encompassed by the present disclosure, and one of skill in the art would readily know how to make, identify, or select such variants or derivatives, and how to test for receptor binding activity of those variants.

In certain non-limiting embodiments, an effective amount or therapeutic dosage of a pharmaceutical composition of the present disclosure contains, sufficient active agent to deliver from about 0.001 μg/kg to about 100 mg/kg (weight of active agent/body weight of the subject). For example, the composition will deliver about 0.01 μg/kg to about 50 mg/kg, and more particularly about 0.1 μg/kg to about 10 mg/kg, and more particularly about 1 μg/kg to about 1 mg/kg. Practice of a method of the present disclosure may comprise administering to a subject an effective amount of the active agent in any suitable systemic and/or local formulation, in an amount effective to deliver the therapeutic dosage of the active agent. In certain embodiments, an effective dosage may be, in a range of about 1 μg/kg to about 1 mg/kg of the active agent.

Practice of the methods of the present disclosure may comprise administering to a subject a therapeutically effective amount of the active agent in any suitable systemic and/or local formulation, in an amount effective to deliver the dosages listed above. The dosage can be administered, for example but not by way of limitation, on a one-time basis, or administered at multiple times (for example but not by way of limitation, from one to five times per day, or once or twice per week), or continuously via a venous drip, depending on the desired therapeutic effect. In one non-limiting example of a therapeutic method of the present disclosure, the dosage is provided in an IV injection or infusion or subcutaneous injection in the range of from about 0.01 mg/kg to about 10 mg/kg of body weight once a day. Alternatively, the active agent may be provided transcutaneously via a slow-release patch applied to the subject's skin.

Administration of the active agent used in the pharmaceutical composition or to practice the method of the present disclosure can be carried out in a variety of conventional ways, such as, but not limited to, orally, by inhalation, vaginally, rectally, topically, nasally, transcutaneously, or by cutaneous-, subcutaneous-, intraperitoneal-, or intravenous-injection. Oral formulations may be formulated such that the active agent passes through a portion of the digestive system before being released, for example it may not be released until reaching the small intestine, or the colon. The active agent may be administered as an eardrop or via a solid or fabric material that can be temporarily inserted into an ear canal.

When an effective amount of the active agent is administered orally, it may be in the form of a solid or liquid preparations such as capsules, pills, tablets, lozenges, melts, powders, suspensions, solutions, elixirs or emulsions. Solid unit dosage forms can be capsules of the ordinary gelatin type containing, for example, surfactants, lubricants, and inert fillers such as lactose, sucrose, and cornstarch, or the dosage forms can be sustained release preparations. The pharmaceutical composition may contain a solid carrier, such as a gelatin or an adjuvant. The tablet, capsule, and powder may contain from about 0.05 to about 95% of the active substance compound by dry weight. When administered in liquid form, a liquid carrier such as water, petroleum, oils of animal or plant origin such as peanut oil, mineral oil, soybean oil, or sesame oil, or synthetic oils may be added. The liquid form of the pharmaceutical composition may further contain physiological saline solution, dextrose or other saccharide solution, or glycols such as ethylene glycol, propylene glycol, or polyethylene glycol. When administered in liquid form, the pharmaceutical composition particularly contains from about 0.005 to about 95% by weight of the active substance. For example, a dose of about 10 mg to about 1000 mg once or twice a day could be administered orally.

In another embodiment, the active agent of the present disclosure can be tableted with conventional tablet bases such as lactose, sucrose, and cornstarch in combination with binders, such as acacia, cornstarch, or gelatin, disintegrating agents such as potato starch or alginic acid, and a lubricant such as stearic acid or magnesium stearate. Liquid preparations are prepared by dissolving the active agent in an aqueous or non-aqueous pharmaceutically acceptable solvent which may also contain suspending agents, sweetening agents, flavoring agents, and preservative agents as are known in the art.

For parenteral administration, for example, the active agent may be dissolved in a physiologically acceptable pharmaceutical carrier and administered as either a solution or a suspension. Illustrative of suitable pharmaceutical carriers are water, saline, dextrose solutions, fructose solutions, ethanol, or oils of animal, vegetative, or synthetic origin. The pharmaceutical carrier may also contain preservatives and buffers as are known in the art.

When an effective amount of the active agent is administered by intravenous, cutaneous, or subcutaneous injection, the compound is particularly in the form of a pyrogen-free, parenterally acceptable aqueous solution or suspension. The preparation of such parenterally acceptable solutions, having due regard to pH, isotonicity, stability, and the like, is well within the skill in the art. A particular pharmaceutical composition for intravenous, cutaneous, or subcutaneous injection may contain, in addition to the active agent, an isotonic vehicle such as Sodium Chloride Injection, Ringer's Injection, Dextrose Injection, Dextrose and Sodium Chloride Injection, Lactated Ringer's Injection, or other vehicle as known in the art. The pharmaceutical compositions of the present disclosure may also contain stabilizers, preservatives, buffers, antioxidants, or other additives known to those of skill in the art.

As noted, particular amounts and modes of administration can be determined by one skilled in the art. One skilled in the art of preparing formulations can readily select the proper form and mode of administration, depending upon the particular characteristics of the active agent selected, the condition to be treated, the stage of the condition, and other relevant circumstances using formulation technology known in the art, described, for example, in Remington: The Science and Practice of Pharmacy, 22^{nd} ed.

Additional pharmaceutical methods may be employed to control the duration of action of the active agent. Increased half-life and/or controlled release preparations may be achieved through the use of proteins or polymers to conjugate, complex with, and/or absorb the active agent as discussed previously herein. The controlled delivery and/or increased half-life may be achieved by selecting appropriate macromolecules (for example but not by way of limitation, polysaccharides, polyesters, polyamino acids, homopolymers, polyvinyl pyrrolidone, ethylenevinylacetate, methylcellulose, or carboxymethylcellulose, and acrylamides such as N-(2-hydroxypropyl) methacrylamide), and the appropriate concentration of macromolecules as well as the methods of incorporation, in order to control release.

Another possible method useful in controlling the duration of action of the active agent by controlled release preparations and half-life is incorporation of the active agent or its functional derivatives into particles of a polymeric material such as polyesters, polyamides, polyamino acids, hydrogels, poly(lactic acid), ethylene vinylacetate copolymers, copolymer micelles of, for example, polyethylene glycol (PEG) and poly(1-aspartamide).

It is also possible to entrap the active agent in microcapsules prepared, for example, by coacervation techniques or by interfacial polymerization (for example, hydroxymethylcellulose or gelatine-microcapsules and poly-(methylmethacylate) microcapsules, respectively), in colloidal drug delivery systems (for example, liposomes, albumin microspheres, microemulsions, nano-particles, and nanocapsules), or in macroemulsions. Such techniques are well known to persons having ordinary skill in the art.

When the active agent is to be used as an injectable material, it can be formulated into a conventional injectable carrier. Suitable carriers include biocompatible and pharmaceutically acceptable phosphate buffered saline solutions, which are particularly isotonic.

For reconstitution of a lyophilized product in accordance with the present disclosure, one may employ a sterile diluent, which may contain materials generally recognized for approximating physiological conditions and/or as required by governmental regulation. In this respect, the sterile diluent may contain a buffering agent to obtain a physiologically acceptable pH, such as sodium chloride, saline, phosphate-buffered saline, and/or other substances which are physiologically acceptable and/or safe for use. In general, the material for intravenous injection in humans should conform to regulations established by the Food and Drug Administration, which are available to those in the field. The pharmaceutical composition may also be in the form of an aqueous solution containing many of the same substances as described above for the reconstitution of a lyophilized product.

The active agent can also be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines, and substituted ethanolamines.

In certain embodiments, the present disclosure includes an active agent composition including: at least one active agent coupled directly or indirectly to a carrier molecule, such as polyethylene glycol (PEG).

### EXAMPLES

Returning now to particular but non-limiting embodiments of the present disclosure, therapeutic functions of GLP-1 agonists in treating acute and progressive hearing damage in both peripheral and central auditory systems in chinchillas exposed to blast exposure treatments were investigated.

Materials and Methods

Healthy young adult chinchillas were used in this study and divided into three groups (7 animals for each): Experimental Groups 1 and 2 and Control Group. GLP-1R agonist (Liraglutide) was delivered to animals with subcutaneous injection at 48 hours before (named as “pre-treatment”) or 2 hours after the blast exposure (named as “post-treatment”) within 7 consecutive days in Exp. Groups 1 and 2, respectively. Each animal was exposed to 3 consecutive blast exposures at the BOP level causing mTBI (15-20 psi or 103-138 kPa) on Day 1 after pre-blast function measurements, including the wideband tympanometry (WBT) for middle ear function, auditory brainstem response (ABR) for healing threshold, distortion product otoacoustic emission (DPOAE) for cochlear function, and middle latency responses (MLRs). The MLRs and ABR were measured to reflect the cortex and subcortical hearing function, respectively. All the central auditory function tests were conducted on Days 1 (4 hours after blast), 4, 7, and 14 for both experiment groups. On the final day of completing all the tests, the animals were euthanized and the brain and cochlea were harvested for histology study to determine the neurophysiology and biomarker changes due to the GLP-1R treatment.

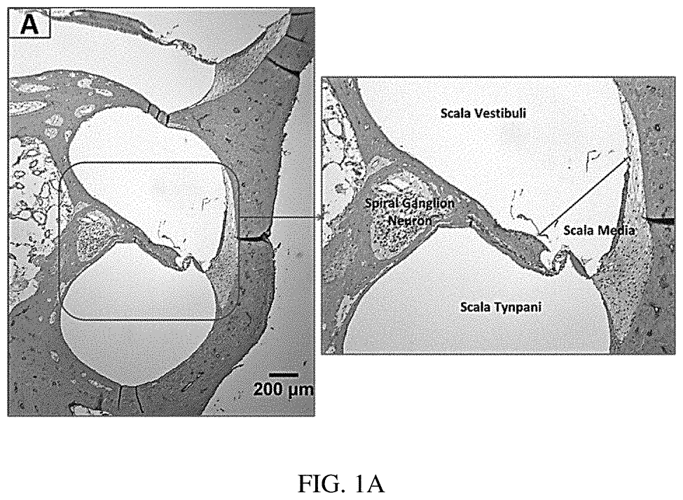

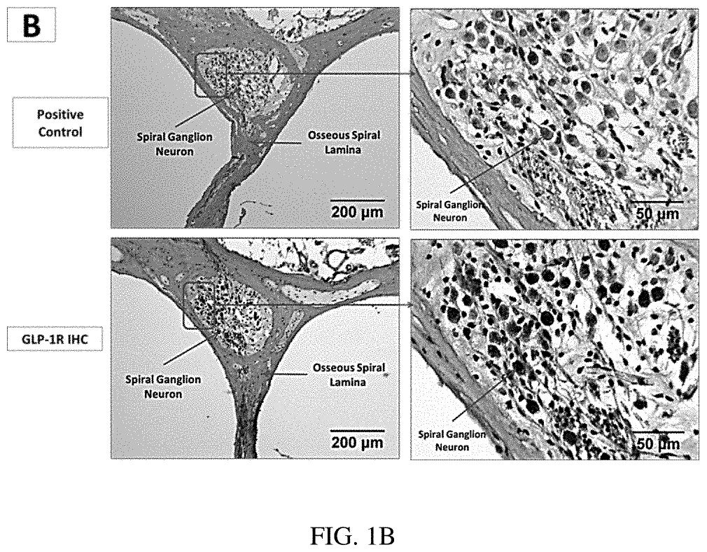

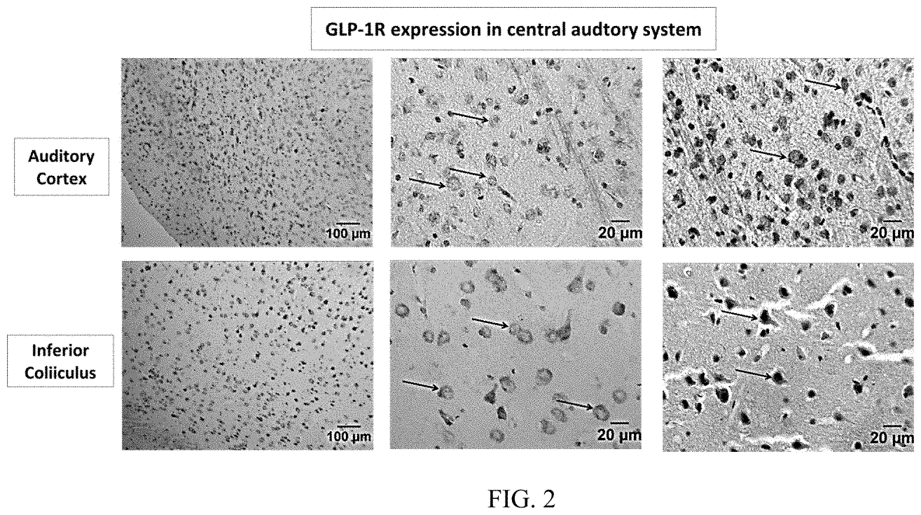

Cochleas harvested from a control animal were analyzed with immunohistochemistry technology to verify the GLP-1R expression in the auditory system of chinchilla. The result in FIG. 1A-B shows that GLP-1R (#188605, Abcam) was widely expressed in the chinchilla's cochlea, especially in the spiral ganglion neurons. Then, we further explored that GLP-1R expression is found in the central auditory system as shown in FIG. 2.

Results

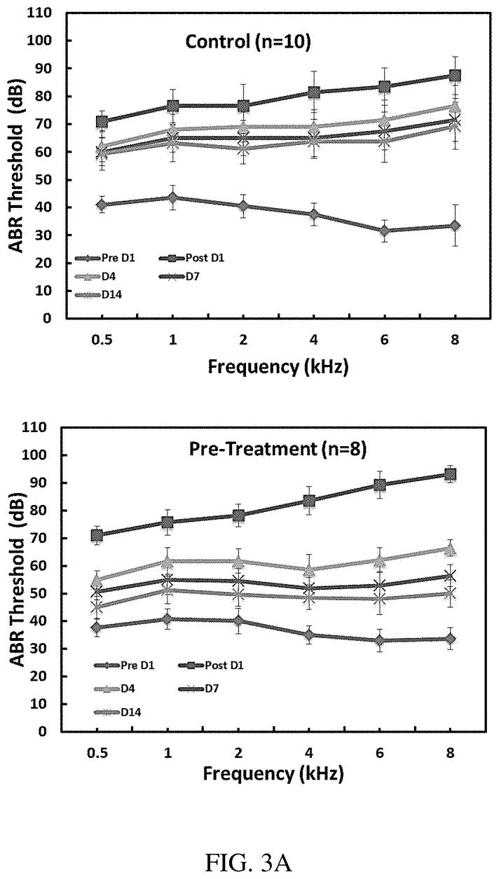

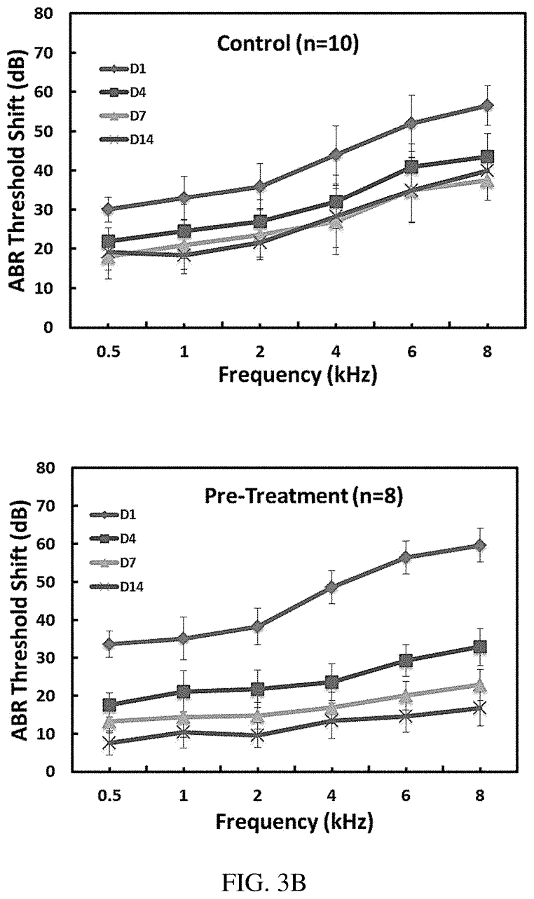

The ABR and DPOAE measurements obtained from the pre-treatment group of chinchillas are shown in FIGS. 3A-5. FIGS. 3A-B displays the mean and standard deviation (SD) of the ABR threshold (FIG. 3A) and threshold shift with respect to the pre-blast (FIG. 3B) across the frequency range of 0.5-8 kHz. The upper and lower left-hand panels represent the results obtained from the control animals (n=10 ears) and the upper and lower right-hand panels represent the results from pre-treatment animals (n=8 ears) after three blasts at mTBI (BOP level at 15-20 psi) on Day 1.

The upper panel of FIG. 3A is the ABR threshold response for the pre-blast (D1), post-blast (D1), D4, D7, and D14. The ABR threshold of post-blast on D1 was linearly increasing with the increase of frequency from 70-88 dB over frequencies from 500 Hz-8 kHz. Following the greatest threshold increase at the post-blast on D1, the ABR threshold decreased on each tested day, but it was almost stable after the large decrease on D4. The upper panel of FIG. 3B clearly displays this trend in showing the ABR threshold shift during the time course from D1 to D14. The greatest shift occurs on D1. After the large shift decrease on D4, each subsequent shift decreases less and there is little difference in the threshold shifts between D7 and D14. On Day 14, an ABR threshold shift of 20 dB at 500 Hz and a 40 dB shift at 8 kHz are observed in control animals.

The lower panels in FIGS. 3A and 3B are ABR results measured from the pre-treatment group. The lower panel of FIG. 3A is the ABR threshold response for the pre-blast (D1), post-blast (D1), D4, D7, and D14. The ABR threshold of post-blast on D1 was also linearly increasing with the increase of frequency from 70-92 dB over frequencies from 500 Hz-8 kHz. Following the greatest threshold increase at the post-blast on D1, the ABR threshold continuously decreased on each tested day after the large decrease on D4. This trend is different from that observed in control group. The lower panel of FIG. 3B clearly displays this trend in showing the ABR threshold shift during the time course from D1 to D14. The greatest shift occurs on D1. After the large decrease on D4, there is a second large decrease on D7 and a continuous decrease to D14. On Day 14, the ABR threshold shift is less than 10 dB at 500 Hz and 15 dB at 8 kHz in the pre-treatment group.

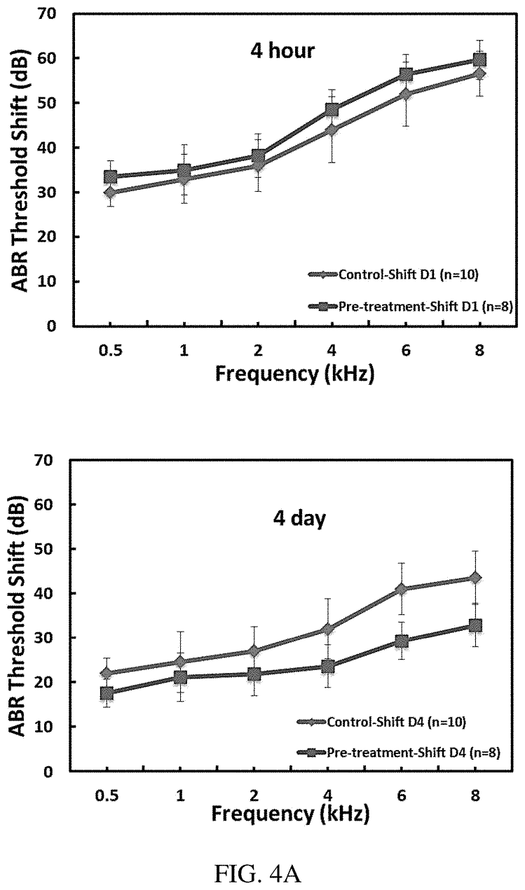

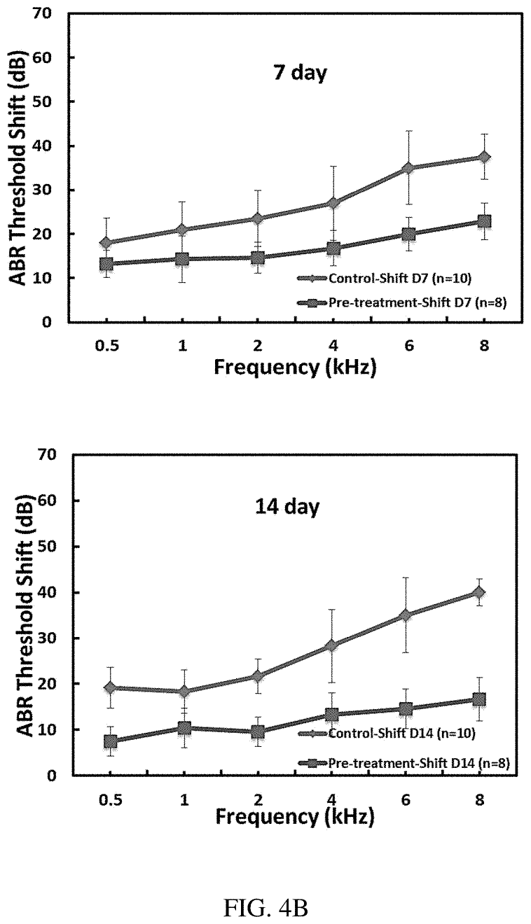

FIGS. 4A-B further display the comparison of ABR threshold shift between the GLP-1 treated group and the control group over the time course: 4 hours (or D1, upper panel of FIG. 4A), 4 days (lower panel, FIG. 4A), 7 days (upper panel of FIG. 4B), and 14 days (lower panel of FIG. 4B). After 4 hours of post-blast, there is not much difference between the ABR shifts in these two groups. As the days extended, the difference is increased across the frequencies from 500 Hz to 8 kHz, and finally on day 14, the ABR threshold shift was 7-15 dB in treatment animals, compared to 20-40 dB in control animals.

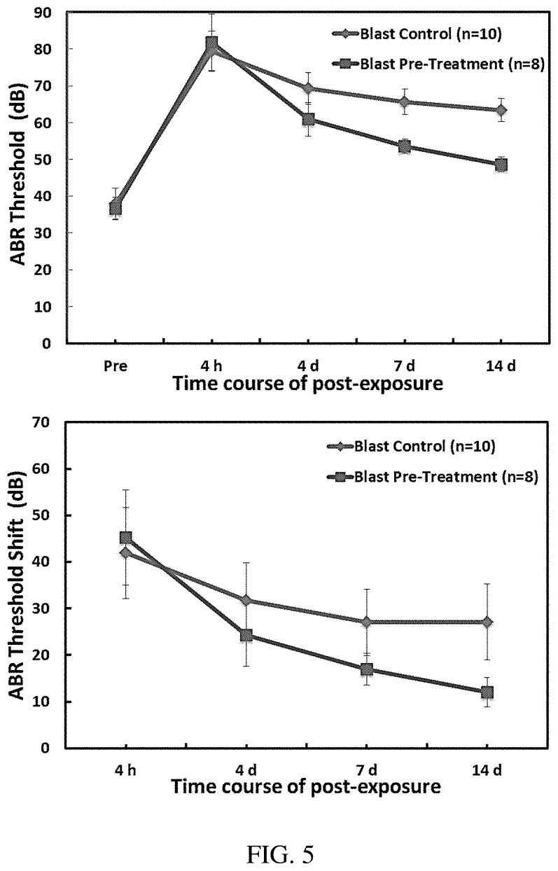

FIG. 5 shows another way to display the different results obtained from the control and treatment groups over the time course using average ABR threshold or threshold shift over the tested frequencies. The hearing restoration was clearly observed in treatment group as days increased. The ABR threshold increased to 80 dB by a shift of 45 dB on Day 1 in both control and pre-treatment groups. After 14 days, the threshold was decreased to 50 dB in treatment group and 65 dB in control group. A 15 dB of hearing recovery was observed in animals pretreated with the GLP-1R agonist.

In conclusion, the hearing restoration induced by the GLP-1R agonist in pre-treatment animals is clearly demonstrated. Animals without GLP-1R agonist treatment and exposure to three times of BOP at the level of equivalent to mTBI resulted in permanent hearing damage with the ABR threshold shift of 20-40 dB over frequencies of 500 Hz-8 kHz after 14 days of post-blast. For animals with the GLP-1R agonist injected subcutaneously at 48 hours before blast exposures, the ABR threshold shift was limited at 7-15 dB over frequencies of 500 Hz-8 kHz after 14 days of post-blast.

The present disclosure is directed to, in at least certain non-limiting embodiments, a method of treating or mitigating hearing loss or auditory impairment in a subject in need of such treatment, comprising: administering to the subject an effective quantity of a glucagon-like peptide-1 receptor (GLP-1R) agonist. The GLP-1R agonist may be, but is not limited to, liraglutide, exenatide, exendin-4, dulaglutide, semaglutide, albiglutide, lixisenatide, taspoglutide, and derivatives thereof. Any other effective GLP-1R agonist may also be used. The hearing loss or auditory impairment may be due to a blast noise (“blast”) or an impulse noise.

The present disclosure is also directed to, in at least certain non-limiting embodiments, a method of prophylactically treating a subject to mitigate potential blast-induced hearing loss, comprising: administering to the subject an effective quantity of a GLP-1R agonist prior to exposure of the subject to a hearing loss-inducing blast, wherein the subject subsequently carries out an assignment during which the subject may be exposed to a blast. The assignment may be a combat mission, or an assignment wherein the subject may be exposed to combat, or a term of duty at a military base or military outpost. The GLP-1R agonist may be, but is not limited to, liraglutide, exenatide, exendin-4, dulaglutide, semaglutide, albiglutide, lixisenatide, taspoglutide, and derivatives thereof. Any other effective GLP-1R agonist may also be used.

The present disclosure is also directed to, in at least certain non-limiting embodiments, a method of treating a subject having a noise-induced hearing loss (NIHL), comprising: administering to the subject an effective quantity of a GLP-1R agonist, wherein the NIHL was caused by exposure of the subject to a blast. For example, the blast may have been caused by an explosion of an improvised explosive device (IED), an explosion of a munition, or a heavy weapon muzzle blast. The GLP-1R may be, but is not limited to, liraglutide, exenatide, exendin-4, dulaglutide, semaglutide, albiglutide, lixisenatide, taspoglutide, and derivatives thereof. Any other effective GLP-1R agonist may also be used.

The present disclosure is directed to, in at least certain non-limiting embodiments, a method of treating a subject for hearing loss or auditory impairment, such as blast-induced hearing damage, comprising administering to the subject in need of such treatment an effective quantity of a GLP-1R agonist. Blast-induced hearing damage may be due to a blast from an improvised explosive device (IED) encountered during a combat mission or in assignment during which combat may have occurred. The method may be prophylactically treating a subject to mitigate potential hearing loss or auditory impairment, such as blast-induced hearing damage, comprising administering to the subject an effective quantity of a GLP-1R agonist prior to exposure of the subject to a hearing loss-inducing blast, wherein the subject then carries out an assignment during which the subject may be exposed to a hearing loss-inducing blast. The assignment may be a combat mission, or an assignment wherein the subject may be exposed to combat. The GLP-1R agonist of the above methods may be, for example, liraglutide, exenatide, exendin-4, dulaglutide, semaglutide, albiglutide, lixisenatide, taspoglutide, or derivatives thereof. Any other effective GLP-1R agonist may also be used.

In certain embodiments, the present disclosure is directed to a GLP-1R agonist for use in treating a subject for hearing loss or auditory impairment, by administering to the subject in need of such treatment an effective quantity of the GLP-1R agonist. The hearing loss or auditory impairment may be blast-induced hearing damage, which may be due to a blast from an improvised explosive device (IED) encountered during a combat mission or in assignment during which combat may have occurred. The GLP-1R agonist may be used to prophylactically treat a subject to mitigate potential hearing loss or auditory impairment, such as blast-induced hearing damage, by administering to the subject an effective quantity of the GLP-1R agonist prior to exposure of the subject to a hearing loss-inducing blast, wherein the subject then carries out an assignment during which the subject may be exposed to a hearing loss-inducing blast. The assignment may be a combat mission, or an assignment wherein the subject may be exposed to combat. The GLP-1R agonist may be, for example, liraglutide, exenatide, exendin-4, dulaglutide, semaglutide, albiglutide, lixisenatide, taspoglutide, and derivatives thereof. Any other effective GLP-1R agonist may also be used. In another embodiment, the disclosure is directed to the application of a GLP-1R agonist (e.g., as listed above) as a treatment for tinnitus, due either to trauma such as by a blast, explosion, or loud noise, chronic exposure of louds noises, or due to a disease condition, e.g., a bacterial infection, or due to a chronic condition such as aging, wherein the GLP-1R agonist may be administered via an ear drop or via a plug of material temporarily inserted into the ear canal.

While the present disclosure has been described in connection with certain embodiments so that aspects thereof may be more fully understood and appreciated, it is not intended that the present disclosure be limited to these particular embodiments. On the contrary, it is intended that all alternatives, modifications and equivalents are included within the scope of the present disclosure. Thus the examples described above, which include particular embodiments, will serve to illustrate the practice of the present disclosure, it being understood that the particulars shown are by way of example and for purposes of illustrative discussion of particular embodiments only and are presented in the cause of providing what is believed to be the most useful and readily understood description of procedures as well as of the principles and conceptual aspects of the presently disclosed methods and compositions. Changes may be made in the formulation of the various compositions described herein, the methods described herein or in the steps or the sequence of steps of the methods described herein without departing from the spirit and scope of the present disclosure.

What is claimed is:

1. A method of treating or mitigating hearing loss or auditory impairment in a subject in need of such treatment, comprising: administering to the subject an effective quantity of a glucagon-like peptide-1 receptor (GLP-1R) agonist.

2. The method of claim 1, wherein the GLP-1R agonist is selected from the group consisting of liraglutide, exenatide, exendin-4, dulaglutide, semaglutide, albiglutide, lixisenatide, taspoglutide, and derivatives thereof.

3. The method of claim 1 wherein the hearing loss or auditory impairment is due to a blast noise.

4. The method of claim 1 wherein the hearing loss or auditory impairment is due to an impulse noise.

5. A method of prophylactically treating a subject to mitigate potential blast-induced hearing loss, comprising: administering to the subject an effective quantity of a glucagon-like peptide-1 receptor (GLP-1R) agonist prior to exposure of the subject to a hearing loss-inducing blast, wherein the subject subsequently carries out an assignment during which the subject may be exposed to a blast.

6. The method of claim 5, wherein the assignment is a combat mission, or an assignment wherein the subject may be exposed to combat.

7. The method of claim 5, wherein the assignment is a term of duty at a military base or military outpost.

8. The method of claim 5, wherein the GLP-1R agonist is selected from the group consisting of liraglutide, exenatide, exendin-4, dulaglutide, semaglutide, albiglutide, lixisenatide, taspoglutide, and derivatives thereof.

9. A method of treating a subject having a noise-induced hearing loss (NIHL), comprising: administering to the subject an effective quantity of a glucagon-like peptide-1 receptor (GLP-1R) agonist, wherein the NIHL was caused by exposure of the subject to a blast.

10. The method of claim 9, wherein the blast was caused by an explosion of an improvised explosive device (IED), an explosion of a munition, or a heavy weapon muzzle blast.

11. The method of claim 9, wherein the GLP-1R agonist is selected from the group consisting of liraglutide, exenatide, exendin-4, dulaglutide, semaglutide, albiglutide, lixisenatide, taspoglutide, and derivatives thereof.
